(19) United States
(12) Patent Application Publication
(10) Pub. No.: US 2013/0023793 A1
(43) Pub. Date: Jan. 24, 2013

(54) SYSTEM AND METHOD FOR MEASURING MID-ARM CIRCUMFERENCE OF A CHILD TO DETERMINE EQUIPMENT AND MEDICATION FOR PEDIATRIC RESUSCITATION

(71) Applicants: Tammy L. Valencia, Aliso Viejo, CA (US); Rowena Bernstein, Huntington Beach, CA (US)

(72) Inventors: Tammy L. Valencia, Aliso Viejo, CA (US); Rowena Bernstein, Huntington Beach, CA (US)

(21) Appl. No.: 13/433,288

(22) Filed: Mar. 29, 2012

Related U.S. Application Data
(60) Provisional application No. 61/510,038, filed on Jul. 20, 2011.

(51) Int. Cl. A61B 5/107

(52) U.S. Cl. 600/587

(57) ABSTRACT

Embodiments of the present invention are directed to a measuring tape for determining equipment sizes and medication dosages to use during the resuscitation of a pediatric patient. In one embodiment, the measuring tape may have a plurality of regions having different colors and/or patterns that identify weight zones that correlate with information and equipment required for resuscitating the pediatric patient. The measuring tape may be wrapped around the arm of the pediatric patient to determine the mid-arm circumference. Once wrapped around the arm, the color and/or pattern in alignment with an opening of the measuring tape may be used to identify a weight zone for the patient which provides the proper equipment size and medication dosages to be used for resuscitating the pediatric patient.

### CLAIM OF PRIORITY

The present Application for Patent claims priority to U.S. Provisional Application No. 61/510,038 entitled “The Broselow Buddy”, filed Jul. 20, 2011, and is hereby expressly incorporated by reference herein.

### FIELD

The present invention relates to a system and method for measuring the arm circumference of a child to determine the proper information, such as equipment and medication, for resuscitating a pediatric patient.

### BACKGROUND

When a patient requires resuscitation, it is critical that interventions, such as defibrillation, drug, and fluid therapy, be started as soon as possible. The proper medications and resuscitation equipment used on the patient are critical as the wrong dosage of medications and the wrong size resuscitation equipment can mean the difference between life and death. With an adult patient, the dosing of medications and the selection of appropriately-sized equipment is relatively straight forward. For example, an adult patient is typically intubated with one of only a few sizes of endotracheal tubes and medical personnel know which size tube to use based on experience. However, when resuscitating a pediatric patient, the interventions are based on weight so pediatric patients require special consideration when calculating medication dosage(s) as well as when selecting equipment. Obtaining a scaled measured weight during a pediatric emergency, when every minute counts, is not an option. Medical personnel, or the resuscitation team, are required to estimate the patient's weight rapidly and accurately.

Currently, the standard practice of determining the weight of a pediatric patient is to use the Broselow® length based tape that is divided along its effective length into color codes which are correlated to the heel-to-crown height of a pediatric patient. A reference at each color code, or color bar, on the tape informs the medical personnel of equipment sizes to perform emergency resuscitation on the pediatric patient. Additionally, a reference at each weight zone, which corresponds to the color code, on the tape shows pre-calculated medication dosages. In an emergency, the Broselow® tape is rolled out flat parallel to the child and the color coded areas directly below the feet inform the medical personnel of the equipment sizes and medication dosages for resuscitating that particular pediatric patient.

The problem with the Broselow® length based tape is that it utilizes only the pediatric patient's height as a predictor of weight and does not take into consideration the prevalence in childhood obesity. Inaccurate weight of a pediatric patient can cause serious overdose or under-dose of medications which may result in the death of the patient.

In view of the above, what is needed is a measuring tape and method that can be used in emergency situations, when it is difficult if not impossible to accurately weigh a patient, which takes into account the variance in body types of children and the rise of childhood obesity when estimating weight.

### SUMMARY OF THE PRESENT INVENTION

In accordance with various aspects of the present invention, a measuring tape for determining equipment sizes and medication dosages to use during the resuscitation of a pediatric patient is provided. In one embodiment, the measuring tape may have a plurality of regions having different colors and/or patterns that identify weight zones that correlate with information and equipment required for resuscitating the pediatric patient. The measuring tape may be wrapped around the arm of the pediatric patient to determine the mid-arm circumference. Once wrapped around the arm, the color and/or pattern in alignment with an opening of the measuring tape may be used to identify a weight zone for the patient which provides the proper equipment size and medication dosages to be used for resuscitating the pediatric patient.

In one embodiment a method for determining equipment and medication for resuscitating a pediatric patient is provided. The method comprises determining a mid-point between a shoulder and elbow of an arm of the pediatric patient is; placing a first portion of a measuring tape at the mid-point and wrapping the measuring tape around the arm such that the measuring tape encircles the arm; identifying a mid-arm circumference of the pediatric patient, the mid-arm circumference identified by a region on the measuring tape that aligns with the first portion when encircling the arm; and correlating the region with a weight zone, the weight zone identifying medication dosages and equipment sizes for resuscitating the pediatric patient.

The first portion of the measuring tape may include an opening and a second portion of the measuring tape may be inserted into and pulled through the opening causing the measuring tape to lay flat on the arm. The region identifying the mid-arm circumference may be in alignment with the opening. Once the region has been determined, a wrist band may be placed around the wrist of the pediatric patient identifying the region. The region may be pre-written on the wrist band or medical personnel may write the region on the wrist band.

In another embodiment a measuring tape for determining the weight of a pediatric patient is provided. The measuring tape comprises a first portion having a generally rectangular shape; a second portion having a generally elongated rectangular shape, the second portion including a first end integrally connected to the first portion and a second end; and an opening, located within the first portion, adapted to receive the second end of the second portion; wherein one side of the second portion includes a plurality of regions identifying different weight zones correlating to weights of pediatric patients; and wherein each of the different weight zones identifies equipment sizes and medication dosages appropriate for the pediatric patients. Each region of the plurality of regions may be identified by a separate color and/or pattern.

The tape may determine the mid-arm circumference of the pediatric patient which may be measured while the patient is standing, sitting or lying down. When measuring the mid-arm circumference, the first portion of the measuring tape may be placed at a mid-point between a shoulder and elbow of an arm of the pediatric patient and then wrapped around the arm and the second portion of the measuring tape is then inserted into and pulled through the opening. The color and/or pattern that are in alignment with the opening may identify a weight zone for the pediatric patient.

According to one aspect of the present invention, the length of the first portion may be approximately one fourth the length of the second portion and the height of the first portion may be approximately double the height of the second portion.

The foregoing, together with other features and advantages of the present invention, will become more apparent when referring to the following specification, claims and accompanying drawings.

### BRIEF DESCRIPTION OF THE DRAWINGS

Various features of the present invention will be better understood from the following detailed description of an exemplary embodiment of the invention, taken in conjunction with the accompanying drawings in which like reference numerals refer to like parts.

FIG. 1 illustrates a measuring tape according to one embodiment of the present invention.

FIG. 2 (comprising FIGS. 2A-2C) illustrates a process of determining a mid-arm circumference of a pediatric patient using a measuring tape.

FIG. 3 illustrates partial information available in a first weight zone.

FIG. 4 illustrates partial information available in a second weight zone.

FIG. 5 illustrates partial information available in a third weight zone.

FIG. 6 illustrates partial information available in a fourth weight zone.

FIG. 7 illustrates partial information available in a fifth weight zone.

FIG. 8 illustrates partial information available in a sixth weight zone.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

In the following description, certain terminology is used to describe certain features of one or more embodiments of the invention. The term “pediatric patient” refers to an infant, child or adolescent seeking medical care. The term “medical personnel” refers to any individual assisting in the resuscitation of a patient, including but not limited to, physicians, nurses, paramedics and emergency medical technicians.

### Overview

Embodiments of the present invention are directed to a measuring tape for determining equipment sizes and medication dosages to use during the resuscitation of a pediatric patient. In one embodiment, the measuring tape may have a plurality of regions having different colors and/or patterns that identify weight zones that correlate with information and equipment required for resuscitating the pediatric patient. The measuring tape may be wrapped around the arm of the pediatric patient to determine the mid-arm circumference. Once wrapped around the arm, the color and/or pattern in alignment with an opening of the measuring tape may be used to identify a weight zone for the patient which provides the proper equipment size and medication dosages to be used for resuscitating the pediatric patient.

### Measuring Tape

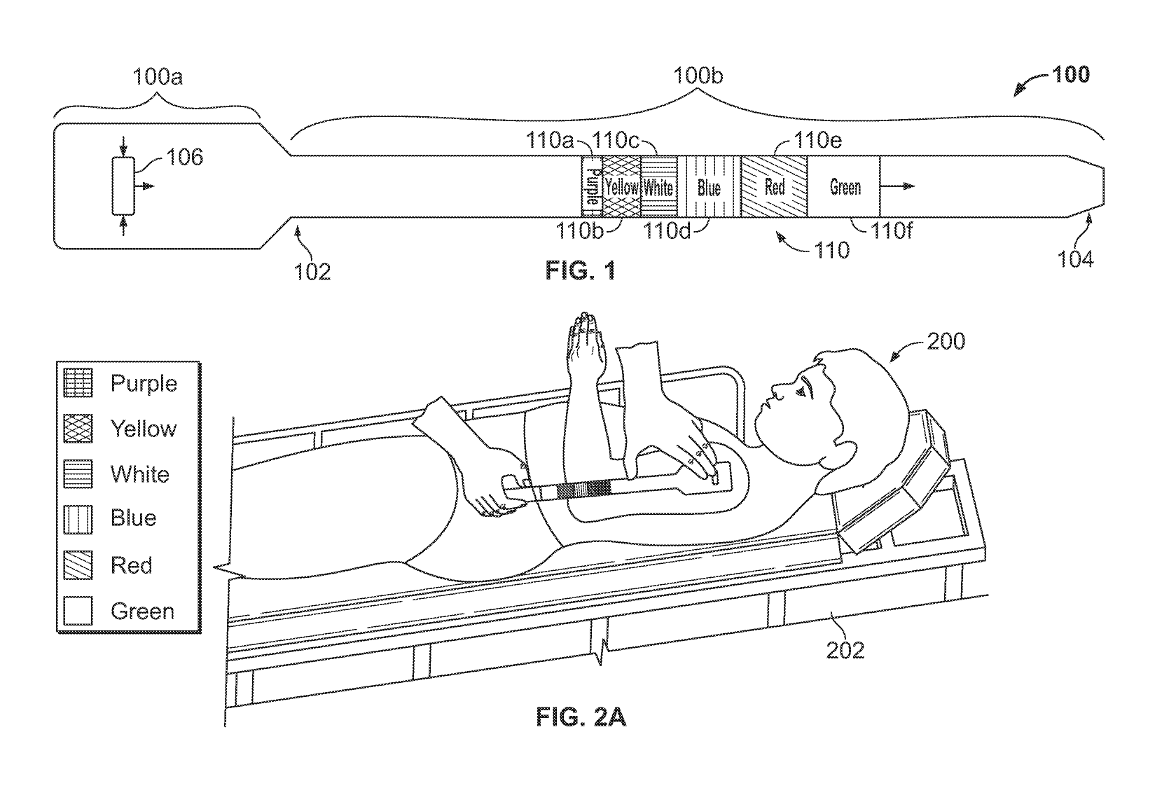

FIG. 1 illustrates a measuring tape according to one embodiment of the present invention. The measuring tape 100 may be used to determine a mid-arm circumference (MAC) of a pediatric patient. As shown, the measuring tape 100 may include a first portion 100a and a second portion 100b, the second portion 100b having a first end 102 integrally connected to the first portion 101a and a second end 104. The first portion 100a may include an opening 106 adapted to receive the second end 104 of the second portion 100b.

According to one embodiment, the first portion 100a may have a generally rectangular shape and the second portion 100a may have a generally elongated rectangular shape. The first portion 100a may have a length approximately a fourth the length of the second portion 100b and a height that is approximately double the height of the second portion 100b. A plurality of regions 110 of different color and/or pattern may be located on the elongated second portion 101b of the measuring tape 100 and each color and/or pattern correspond to a different weight zone. Pre-defined weight zones may provide pre-calculated medication dosages and equipment sizes. That is, the possible weights of a child may be divided into ranges and each range may represent a weight zone. In one example, the weight zones may be the same as or correlate with the weight zones identified on the Broselow® pediatric emergency tape. In one example, the colors may include purple 110a, yellow 110b, white 110c, blue 110d, red 110e and green 110f. The measuring tape 100 may be used to immediately provide medical personnel an estimated weight (i.e. MAC estimated weight).

### Mid-Arm Circumference Measurement

The mid-arm circumference (MAC) takes into account the pediatric patient's body type and has been shown to be a valid measurement from which to derive a pediatric patient's width. It has been established that the following formula may accurately derive a pediatric patient's weight:

Pediatric patient weight (kg)=(MAC(cm)×3.5)−30

In an emergency situation, such as cardiac arrest, when stress levels are high and there is not time for double checking, there is a high risk for error or miscalculation using this formula. The measuring tape 100 of FIG. 1 can provide an accurate estimated weight of the pediatric patient and eliminate the need for formula calculation and as a result eliminating the error or miscalculation.

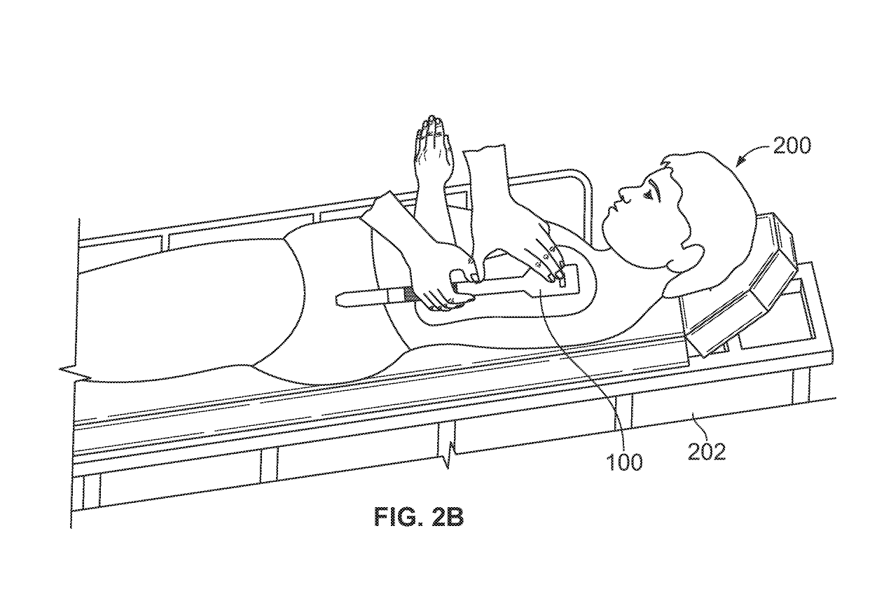

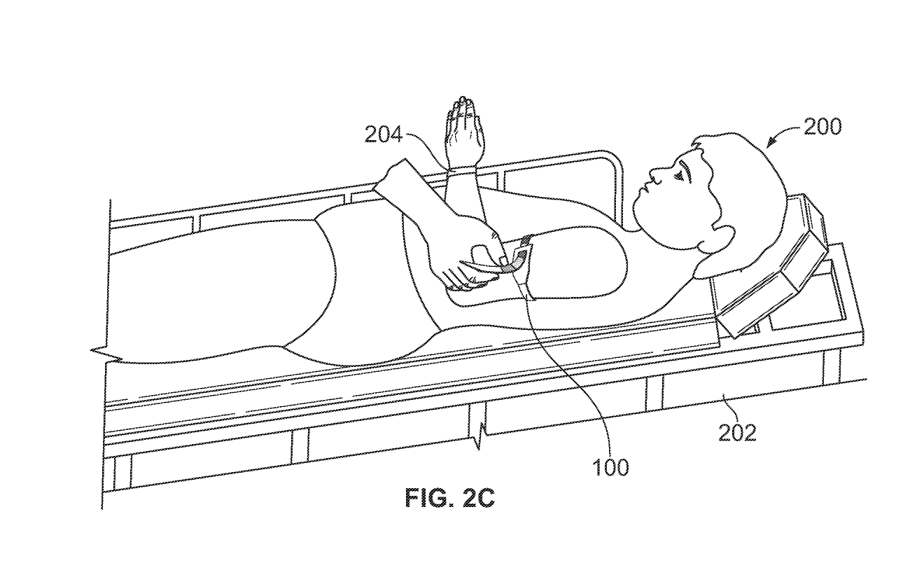

When a pediatric patient is brought into an emergency area the patient may be sitting standing or lying down. Unlike the Broselow® pediatric emergency tape, the patient does need to be lying down. A health care worker, such as a nurse, may eyeball the approximate mid-point between the shoulder and the elbow on the arm of the pediatric patient. If there is any clothing or other material covering the arm this clothing should be removed. Next, the health care worker may place the first portion 100a of the measuring tape 100 at this mid-point and then wrap the measuring tape 100 around the arm of the patient inserting the second end 100b into the opening 104 such that the measuring tape 100 is encircling a portion of the patient's upper arm lying flat on the skin. The region (i.e. color and/or pattern) in alignment with the opening 106 may then be used to correlate with a weight zone, described below in further detail, which identifies information (such as proper medication dosages) and equipment required to resuscitate a pediatric patient. A chart may be used to identify a plurality of different weight zones. According to one example, the weight zones may be similar or identical to the color categories located on the Broselow® pediatric emergency tape. Once the weight zone for the pediatric patient has been identified, a wrist band 204 identifying the weight zone may be placed on the wrist of the patient (See FIG. 2C) preventing the need to continually re-determine the patient's weight. The wrist band 204 may be color coded to match the weight zone or the wrist band may be blank and medical personnel write the color or pattern on the band. The wristbands may be made from Tyvek®, a strong water resistant material made by Dupont®. The ends of the wristband 204 may include a waterproof adhesive allowing the wristband 204 to be easily placed around the wrist of the patient. In one embodiment, the wristband 204 may have a width of ¾ inch (19 mm) and a length of 10 inches (25 cm).

Turning to FIG. 2 (comprising FIGS. 2A-2C), a typical process of determining a mid-arm circumference of a pediatric patient, in a non-emergency situation, using the measuring tape of FIG. 1 is illustrated. As shown in FIG. 2A, a pediatric patient 200 may be lying on a gurney 202 although the measurement can also be accurately determined if the patient is standing or sitting. For the most accurate measurement, any clothing covering the arms of the patient should be removed. Medical personnel may then bend the arm of the patient at the elbow so that it is at approximately a 90 degree angle. Next, the location of the arm in which to take the measurement (i.e. the mid-arm circumference) may be found by locating the middle of the left upper arm of the pediatric patient which is located between the tip of the shoulder and the tip of the elbow. The first portion 100a of the tape 100 may then be placed at the middle of the left upper arm and wrapped around the arm at the midpoint with its color coding right side up. The second end 104 of the second portion 100b of the tape 100 may be inserted into and pulled through the opening 106 so that the tape 100 lays flat on the skin. The tape 100 should not be pulled so tight that it notches the skin nor should the tape 100 be too loose so that the tape 100 loses its contact with the skin. When the tape 100 is in the correct position on the arm with the correct tension, the color and/or pattern that is in alignment with the opening 106 may then be used to correlate with a weight zone which identifies the proper medication dosages and equipment that should be used on the patient.

### Weight Zones

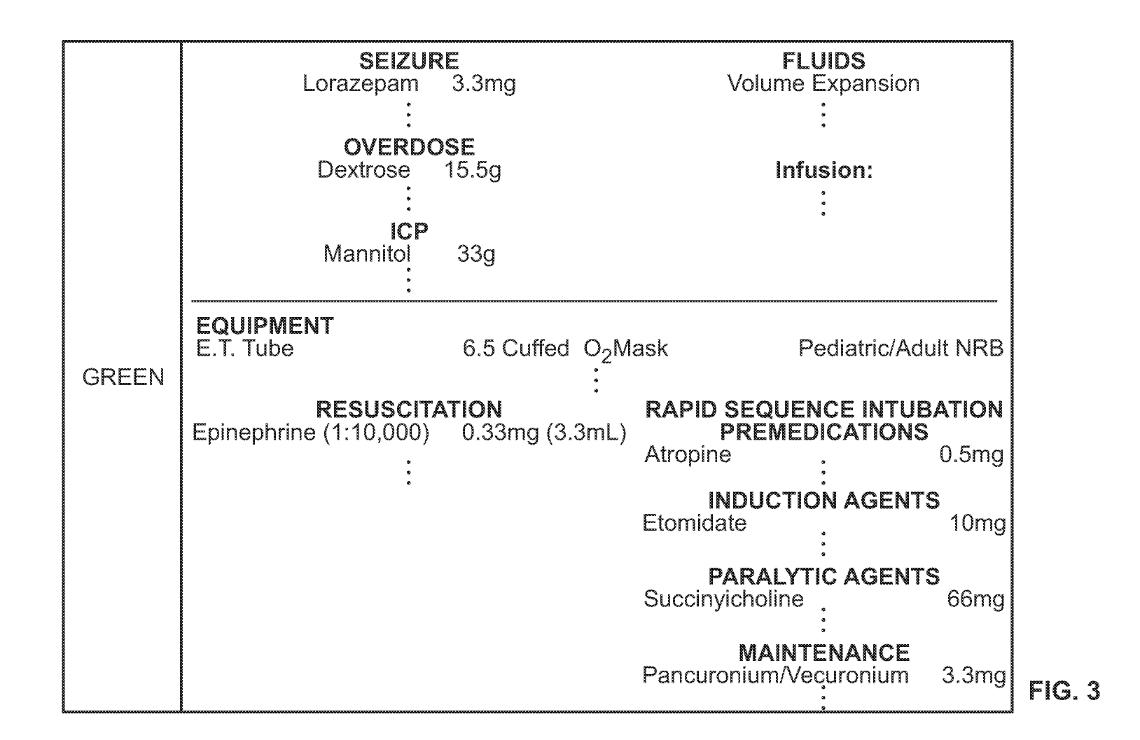

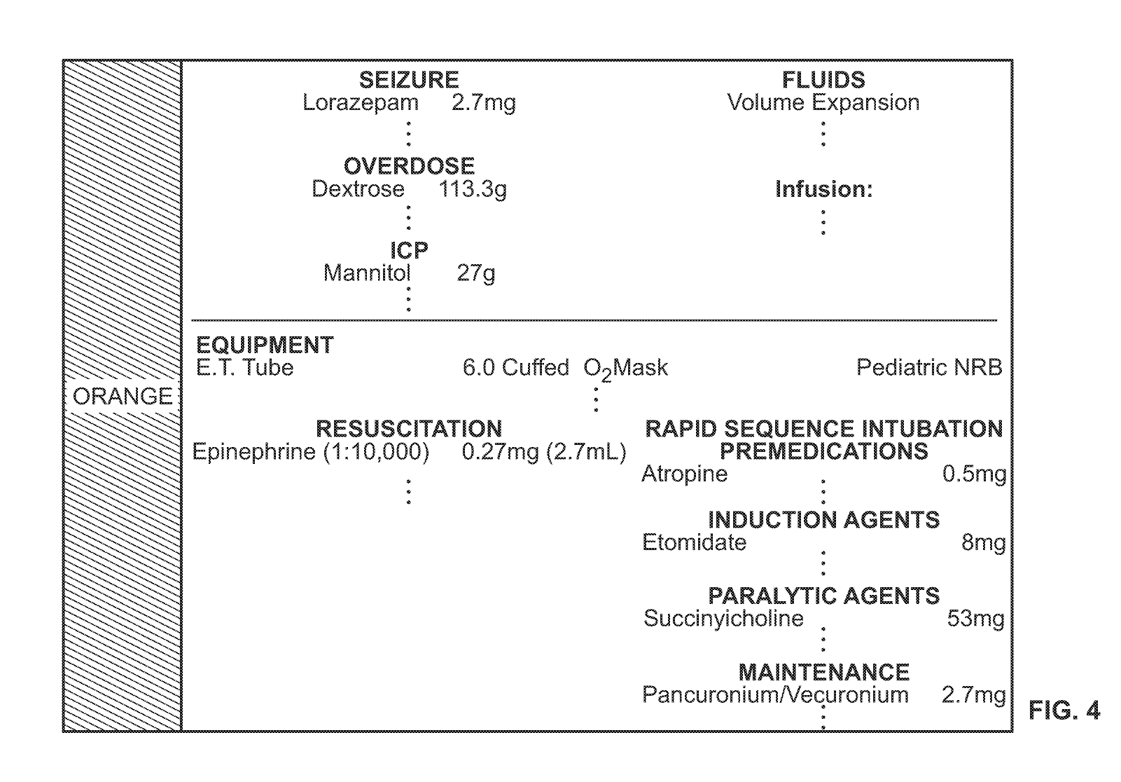

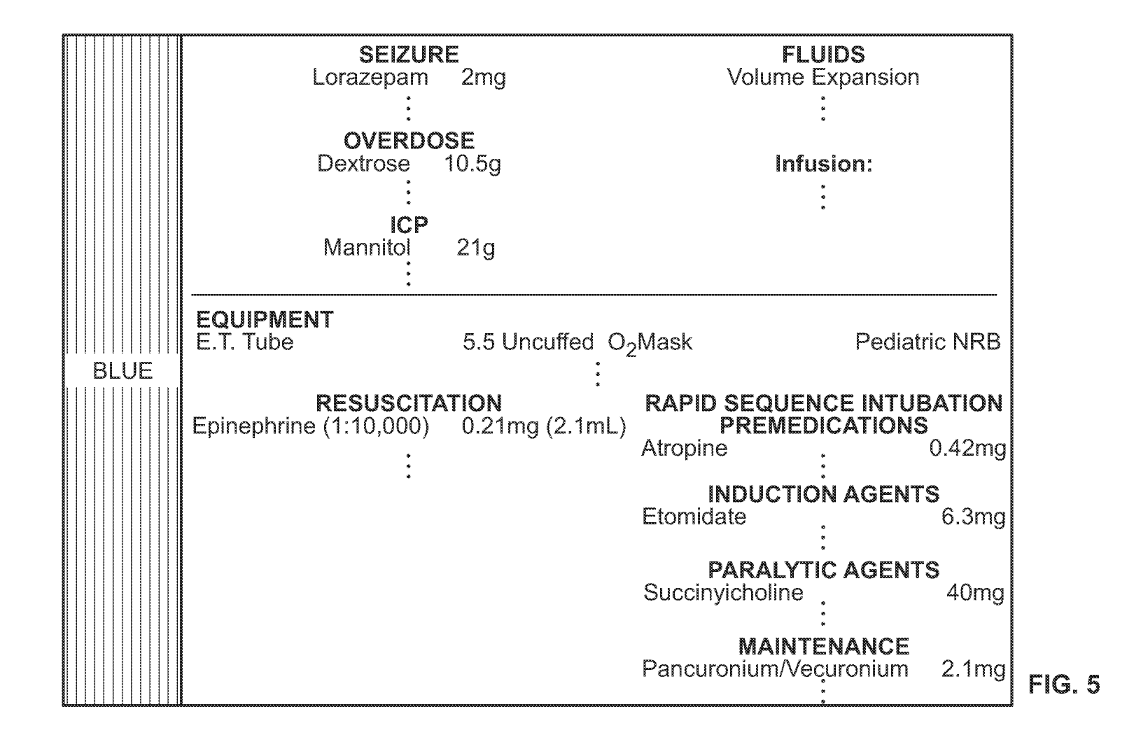

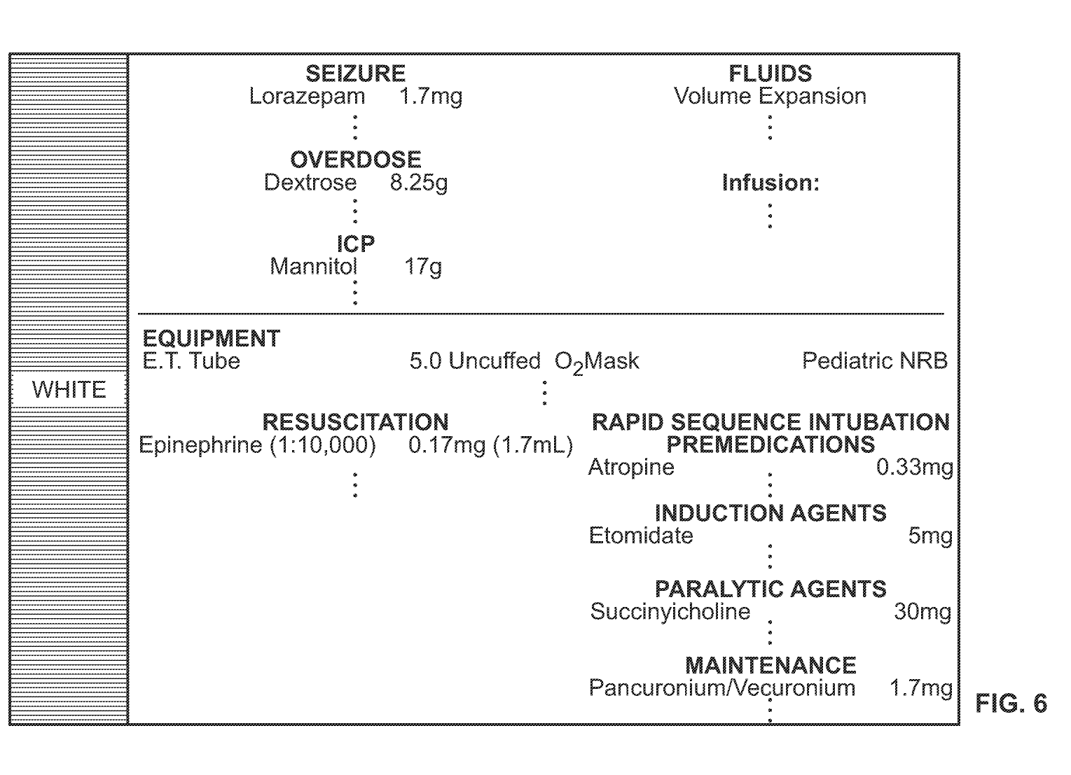

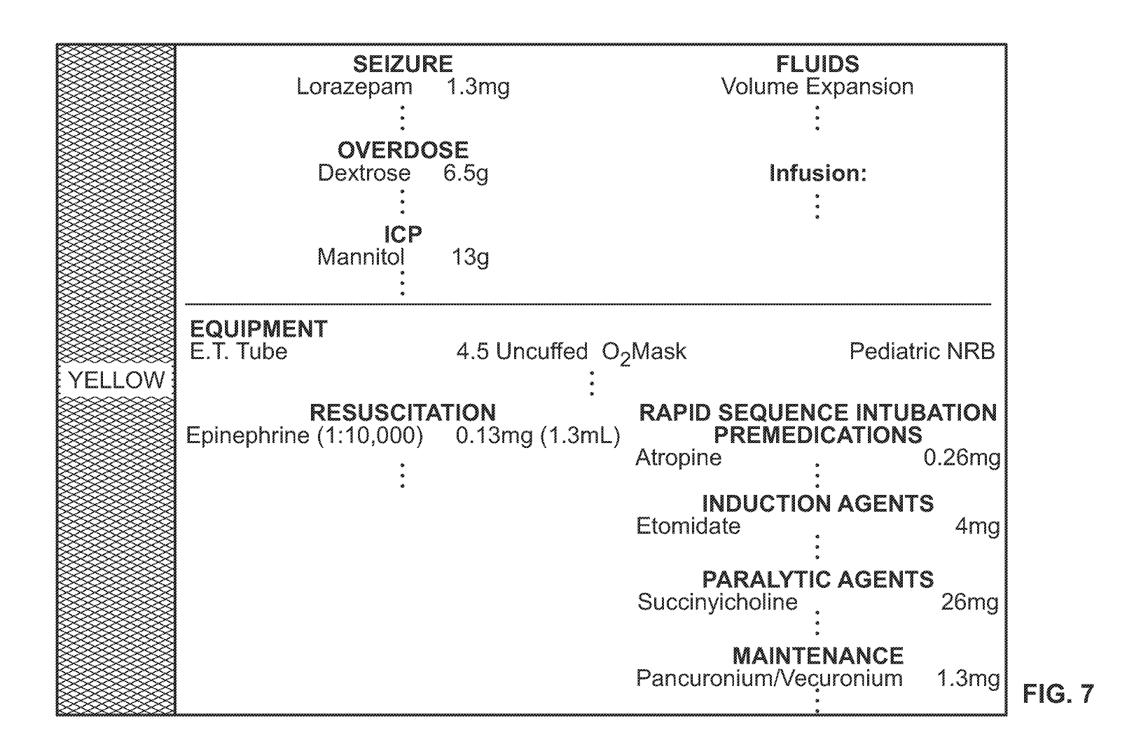

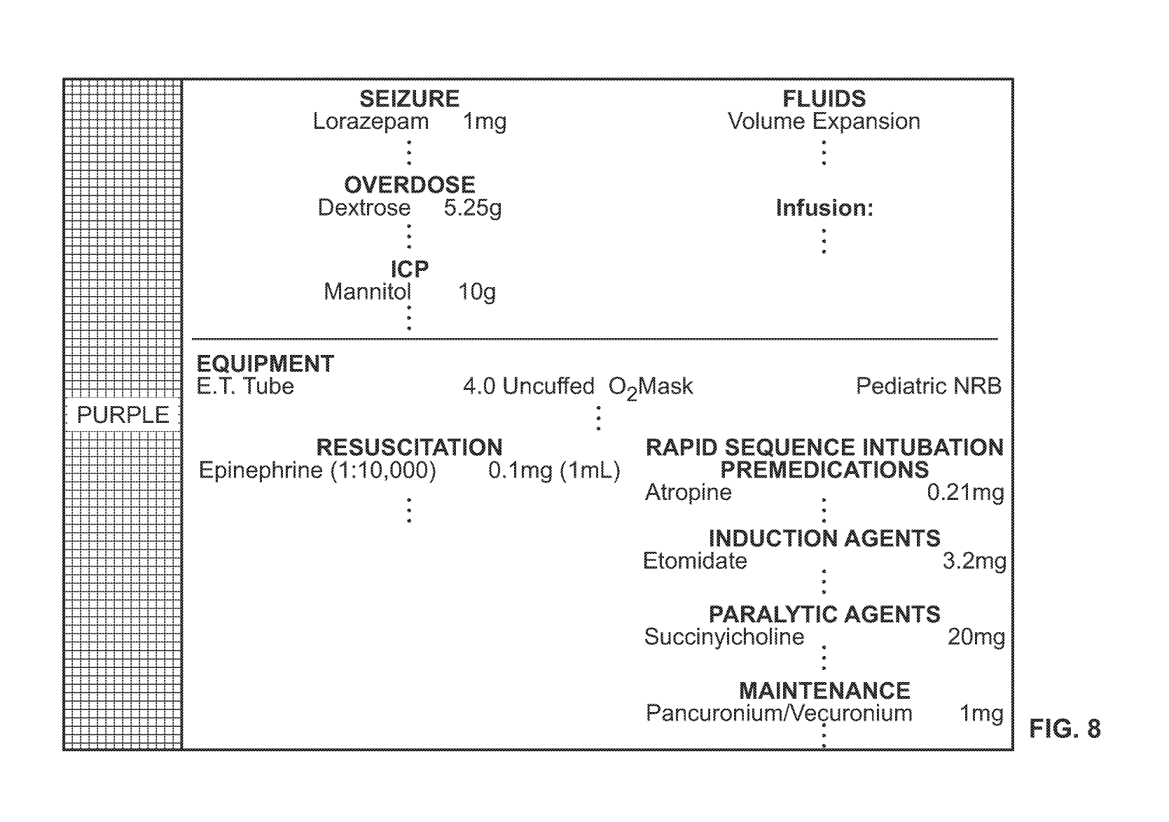

As described above, pre-defined weight zones may provide pre-calculated medication dosages and equipment sizes. That is, the possible weights of a child may be divided into ranges and each range may represent a weight zone. FIGS. 3-8 illustrate partial information available in a plurality of weight zones. In one embodiment, the weight zones may correspond to the weight zones identified on the Broselow® pediatric emergency tape. For example, the first weight zone shown in FIG. 3 may correspond with the weight zone identified by the green section of the Broselow® pediatric emergency tape; the second weight zone shown in FIG. 4 may correspond with the weight zone identified by the orange section of the Broselow® pediatric emergency tape; the third weight zone shown in FIG. 5 may correspond with the weight zone identified by the blue section of the Broselow® pediatric emergency tape; the fourth weight zone shown in FIG. 6 may correspond with the weight zone identified by the white section of the Broselow® pediatric emergency tape; the fifth weight zone shown in FIG. 7 may correspond with the weight zone identified by the yellow section of the Broselow® pediatric emergency tape; and the sixth weight zone shown in FIG. 8 may correspond with the weight zone identified by the purple section of the Broselow® pediatric emergency tape.

Illustrated below are tables which identify all the available information in the plurality of weight zones. Table 1 illustrates the weight zone identified by the green section of the Broselow® pediatric emergency tape. Table 2 illustrates the weight zone identified by the orange section of the Broselow® pediatric emergency tape. Table 3 illustrates the weight zone identified by the blue section of the Broselow® pediatric emergency tape. Table 4 illustrates the weight zone identified by the white section of the Broselow® pediatric emergency tape. Table 5 illustrates the weight zone identified by the yellow section of the Broselow® pediatric emergency tape. Table 6 illustrates the weight zone identified by the purple section of the Broselow® pediatric emergency tape.

According to one example, a pediatric patient determined to have a weight within the weight zone identified by the green section of the Broselow® pediatric emergency tape would require a 32-33 chest tube and 33 mEq of Sodium Bicarbonate.

TABLE 1
First Weight Zone
GREEN
SEIZURE

| | | |
|---|---|---|
| Lorazepam | 3.3 | mg |
| Diazepam IV | 6.6 | mg |
| Diazepam-RECTAL | 10 | mg |
| Phenobarbital Load | 660 | mg |
| Phenytoin Load | 500 | mg |
| Fosphenytoin Load | 500 | mg-PE |

OVERDOSE

| | | |
|---|---|---|
| Dextrose | 16.5 | g |
| Naloxone | 2 | mg |
| Flumazenil | 0.2 | mg |
| Glucagon | 1 | mg |
| Charcoal | 33 | g |

ICP

| | | |
|---|---|---|
| Mannitol | 33 | g |
| Furosemide | 33 | mg |

FLUIDS

| | | |
|---|---|---|
| Volume Expansion | | |
| Crystalloid (NS or LR) | 640 | mL |
| Colloid, Blood | 330 | mL |
| Maintenance | | |
| DSW + 1/2 NS + 29 | 73 | mL/HR |

Infusion:
Pursuant to JCAHO's
National Patient Safety Goal 3b-
“Rule of 6” for infusions
Should be converted to
Standardized Concentrations.

| Equipment | | |
|---|---|---|
| E.T Tube | 6.5 Cuffed | |
| E.T Insertion Length | 18.5-19.5 | cm |
| Stylet | 14 | French |
| Suction Catheter | 10-12 | French |
| Laryngoscope | 3 Straight or Curved | |
| BVM | Adult | |
| Oral Airway | 80 | mm |
| Nasopharyngeal Airway | 30 | French |
| LMA | 3 | |
| O₂ Mask | Pediatric/Adult NR8 | |
| ETCO₂ | Adult | |
| Urinary Catheter | 12 | French |
| Chest Tube | 32-33 | French |
| NG Tube | 16-18 | French |
| Vascular Access | 16-20 | Ga |
| Intraosseous | 15 | Ga |

| | |
|---|---|
| BP Cuff | Small Adult |

RESUSCITATION

| Epinephrine (1:10,000) | 0.33 mg | (3.3 mL) |
|---|---|---|
| Epinephrine ET (1:1,000) | 3.3 mg | (3.3 mL) |
| Atropine (0.1 mg/mL) | 0.5 mg | (5 mL) |
| Atropine ET (0.4 mg/mL) | 1 mg | (2.5 mL) |
| Sodium Bicarbonate | 33 | mEq |
| Lidocaine | 33 | mg |
| Lidocaine ET | 66-100 | mgs |
| Defibrillation | | |
| 1^{st}/2^{nd} Dose (may repeat) | 66 J/132 J | |
| Cardioversion | | |
| 1^{st}/2^{nd} Dose | 33 J/66 J | |
| Adenosine | | |
| Ist Dose | 3.3 | mg |
| 2^{nd} Dose If Needed | 6.6 | mg |
| Amiodarone | 165 | mg |
| Calcium Chloride | 660 | mg |
| Magnesium Sulfate | 1650 | mg |

RAPID SEQUENCE INTUBATION
PREMDICATIONS

| | | |
|---|---|---|
| Atropine | 0.5 | mg |
| Pan/Vecuronium | | |
| (Defasiculating Agent) | 0.33 | mg |
| Lidocaine | 50 | mg |
| Fentanyl | 100 | mog |

INDUCTION AGENTS

| | | |
|---|---|---|
| Etomidale | 10 | mg |
| Ketamine | 66 | mg |
| Midazolam | 10 | mg |
| Propofol | 100 | mg |

PARALYTIC AGENTS

| | | |
|---|---|---|
| Succinylcholine | 66 | mg |
| Pancuronium | 6.6 | mg |
| Vecuronium | 6.6 | mg |
| Rocuronium | 33 | mg |

MAINTENANCE

| | | |
|---|---|---|
| Pancuronium/Vecuronium | 3.3 | mg |
| Lorazepam | 1.7 | mg |

TABLE 2
Second Weight Zone
ORANGE
SEIZURE

| | | |
|---|---|---|
| Lorazepam | 2.7 | mg |
| Diazepam IV | 5.3 | mg |
| Diazepam-RECTAL | 8 | mg |
| Phenobarbital Load | 530 | mg |
| Phenytoin Load | 400 | mg |
| Fosphenytoin Load | 400 | mg-PE |

OVERDOSE

| | | |
|---|---|---|
| Dextrose | 13.3 | g |
| Naloxone | 2 | mg |
| Flumazenil | 0.2 | mg |
| Glucagon | 1 | mg |
| Charcoal | 27 | g |

ICP

| | | |
|---|---|---|
| Mannitol | 27 | g |
| Furosemide | 27 | mg |

FLUIDS

| | | |
|---|---|---|
| Volume Expansion | | |
| Crystalloid (NS or LR) | 530 | mL |
| Colloid, Blood | 270 | mL |
| Maintenance | | |
| DSW + 1/2 NS + 29 | 68 | mL/HR |

Infusion:
Pursuant to JCAHO's
National Patient Safety Goal 3b-
“Rule of 6” for infusions
Should be converted to
Standardized Concentrations.

| Equipment | | |
|---|---|---|
| E.T Tube | 6.0 Cuffed | |
| E.T Insertion Length | 17-18 | cm |
| Stylet | 14 | French |
| Suction Catheter | 10 | French |
| Laryngoscope | 2 Straight or Curved | |
| *BVM | Child | |
| Oral Airway | 80 | mm |
| Nasopharyngeal Airway | 26 | French |
| LMA | 2.5 | |
| O₂ Mask | Pediatric NR8 | |
| ETCO₂ | Adult | |
| Urinary Catheter | 12 | French |
| Chest Tube | 28-32 | French |
| NG Tube | 14-18 | French |
| Vascular Access | 18-20 | Ga |
| Intraosseous | 15 | Ga |

| | |
|---|---|
| BP Cuff | Child |

RESUSCITATION

| Epinephrine (1:10,000) | 0.27 mg | (2.7 mL) |
|---|---|---|
| Epinephrine ET (1:1,000) | 2.7 mg | (2.7 mL) |
| Atropine (0.1 mg/mL) | 0.5 mg | (5 mL) |
| Atropine ET (0.4 mg/mL) | 0.8 mg | (2 mL) |
| Sodium Bicarbonate | 27 | mEq |
| Lidocaine | 27 | mg |
| Lidocaine ET | 54-80 | mgs |
| Defibrillation | | |
| 1^{st}/2^{nd} Dose (may repeat) | 53 J/106 J | |
| Cardioversion | | |
| 1^{st}/2^{nd} Dose | 27 J/53 J | |
| Adenosine | | |
| Ist Dose | 2.7 | mg |
| 2^{nd} Dose If Needed | 5.4 | mg |
| Amiodarone | 130 | mg |
| Calcium Chloride | 530 | mg |
| Magnesium Sulfate | 1325 | mg |

RAPID SEQUENCE INTUBATION
PREMDICATIONS

| | | |
|---|---|---|
| Atropine | 0.5 | mg |
| Pan/Vecuronium | | |
| (Defasiculating Agent) | 0.27 | mg |
| Lidocaine | 40 | mg |
| Fentanyl | 80 | mcg |

INDUCTION AGENTS

| | | |
|---|---|---|
| Etomidale | 8 | mg |
| Ketamine | 50 | mg |
| Midazolam | 8 | mg |
| Propofol | 80 | mg |

PARALYTIC AGENTS

| | | |
|---|---|---|
| Succinylcholine | 53 | mg |
| Pancuronium | 5.3 | mg |
| Vecuronium | 5.3 | mg |
| Rocuronium | 27 | mg |

MAINTENANCE

| | | |
|---|---|---|
| Pancuronium/Vecuronium | 2.7 | mg |
| Lorazepam | 1.3 | mg |

TABLE 3
Third Weight Zone
BLUE
SEIZURE

| | | |
|---|---|---|
| Lorazepam | 2 | mg |
| Diazepam IV | 4.2 | mg |
| Diazepam-RECTAL | 10 | mg |
| Phenobarbital Load | 420 | mg |
| Phenytoin Load | 315 | mg |
| Fosphenytoin Load | 315 | mg-PE |

OVERDOSE

| | | |
|---|---|---|
| Dextrose | 10.5 | g |
| Naloxone | 2 | mg |
| Flumazenil | 0.2 | mg |
| Glucagon | 1 | mg |
| Charcoal | 21 | g |

ICP

| | | |
|---|---|---|
| Mannitol | 21 | g |
| Furosemide | 21 | mg |

FLUIDS

| | | |
|---|---|---|
| Volume Expansion | | |
| Crystalloid (NS or LR) | 420 | mL |
| Colloid, Blood | 210 | mL |
| Maintenance | | |
| DSW + 1/2 NS + 29 | 63 | mL/HR |

Infusion:
Pursuant to JCAHO's
National Patient Safety Goal 3b-
“Rule of 6” for infusions
Should be converted to
Standardized Concentrations.

| Equipment | | |
|---|---|---|
| E.T Tube | 5.5 Uncuffed | |
| E.T Insertion Length | 16.5 | cm |
| Stylet | 14 | French |
| Suction Catheter | 10 | French |
| Laryngoscope | 2 Straight or Curved | |
| BVM | Child | |
| Oral Airway | 70 | mm |
| Nasopharyngeal Airway | 24 | French |
| LMA | 2-2.5 | |
| O₂ Mask | Pediatric NR8 | |
| ETCO₂ | Adult | |
| Urinary Catheter | 10-12 | French |
| Chest Tube | 24-32 | French |
| NG Tube | 12-14 | French |
| Vascular Access | 18-20 | Ga |
| Intraosseous | 15 | Ga |

| | |
|---|---|
| BP Cuff | Child |

RESUSCITATION

| Epinephrine (1:10,000) | 0.21 mg | (2.1 mL) |
|---|---|---|
| Epinephrine ET (1:1,000) | 2.1 mg | (2.1 mL) |
| Atropine (0.1 mg/mL) | 0.42 mg | (4.2 mL) |
| Atropine ET (0.4 mg/mL) | 0.6 mg | (1.5 mL) |
| Sodium Bicarbonate | 21 | mEq |
| Lidocaine | 20 | mg |
| Lidocaine ET | 40-60 | mgs |
| Defibrillation | | |
| 1^{st}/2^{nd} Dose (may repeat) | 40 J/80 J | |
| Cardioversion | | |
| 1^{st}/2^{nd} Dose | 20 J/40 J | |
| Adenosine | | |
| Ist Dose | 2.1 | mg |
| 2^{nd} Dose If Needed | 4.2 | mg |
| Amiodarone | 105 | mg |
| Calcium Chloride | 420 | mg |
| Magnesium Sulfate | 1050 | mg |

RAPID SEQUENCE INTUBATION
PREMDICATIONS

| | | |
|---|---|---|
| Atropine | 0.42 | mg |
| Pan/Vecuronium | | |
| (Defasiculating Agent) | 0.21 | mg |
| Lidocaine | 32 | mg |
| Fentanyl | 63 | mcg |

INDUCTION AGENTS

| | | |
|---|---|---|
| Etomidale | 6.3 | mg |
| Ketamine | 42 | mg |
| Midazolam | 6.3 | mg |
| Propofol | 63 | mg |

PARALYTIC AGENTS

| | | |
|---|---|---|
| Succinylcholine | 40 | mg |
| Pancuronium | 4.2 | mg |
| Vecuronium | 4.2 | mg |
| Rocuronium | 21 | mg |

MAINTENANCE

| | | |
|---|---|---|
| Pancuronium/Vecuronium | 2.1 | mg |
| Lorazepam | 1 | mg |

TABLE 4
Fourth Weight Zone
WHITE
SEIZURE

| | | |
|---|---|---|
| Lorazepam | 1.7 | mg |
| Diazepam IV | 3.3 | mg |
| Diazepam-RECTAL | 8 | mg |
| Phenobarbital Load | 330 | mg |
| Phenytoin Load | 250 | mg |
| Fosphenytoin Load | 250 | mg-PE |

OVERDOSE

| | | |
|---|---|---|
| Dextrose | 8.25 | g |
| Naloxone | 1.6 | mg |
| Flumazenil | 0.16 | mg |
| Glucagon | 0.5 | mg |
| Charcoal | 16.5 | g |

ICP

| | | |
|---|---|---|
| Mannitol | 17 | g |
| Furosemide | 17 | mg |

FLUIDS

| | | |
|---|---|---|
| Volume Expansion | | |
| Crystalloid (NS or LR) | 325 | mL |
| Colloid, Blood | 165 | mL |
| Maintenance | | |
| DSW + 1/2 NS + 29 | 55 | mL/HR |

Infusion:
Pursuant to JCAHO's
National Patient Safety Goal 3b-
“Rule of 6” for infusions
Should be converted to
Standardized Concentrations.

| Equipment | | |
|---|---|---|
| E.T Tube | 5.0 Uncuffed | |
| E.T Insertion Length | 14-15 | cm |
| Stylet | 6 | French |
| Suction Catheter | 10 | French |
| Laryngoscope | 2 Straight | |
| BVM | Child | |
| Oral Airway | 60 | mm |
| Nasopharyngeal Airway | 22 | French |
| LMA | 2 | |
| O₂ Mask | Pediatric NR8 | |
| ETCO₂ | Adult | |
| Urinary Catheter | 10-12 | French |
| Chest Tube | 20-24 | French |
| NG Tube | 10 | French |
| Vascular Access | 18-22 | Ga |
| Intraosseous | 15 | Ga |

| | |
|---|---|
| BP Cuff | Child |

RESUSCITATION

| Epinephrine (1:10,000) | 0.17 mg | (1.7 mL) |
|---|---|---|
| Epinephrine ET (1:1,000) | 1.7 mg | (1.7 mL) |
| Atropine (0.1 mg/mL) | 0.33 mg | (3.3 mL) |
| Atropine ET (0.4 mg/mL) | 0.5 mg | (1.2 mL) |
| Sodium Bicarbonate | 16.5 | mEq |
| Lidocaine | 17 | mg |
| Lidocaine ET | 34-50 | mgs |
| Defibrillation | | |
| 1^{st}/2^{nd} Dose (may repeat) | 33 J/66 J | |
| Cardioversion | | |
| 1^{st}/2^{nd} Dose | 17 J/33 J | |
| Adenosine | | |
| Ist Dose | 1.7 | mg |
| 2^{nd} Dose If Needed | 3.3 | mg |
| Amiodarone | 80 | mg |
| Calcium Chloride | 330 | mg |
| Magnesium Sulfate | 820 | mg |

RAPID SEQUENCE INTUBATION
PREMDICATIONS

| Atropine | 0.33 | mg |
|---|---|---|
| Pan/Vecuronium | N/A | |
| (Defasiculating Agent) | N/A < 20 kg | |
| Lidocaine | 25 | mg |
| Fentanyl | 50 | mcg |

INDUCTION AGENTS

| | | |
|---|---|---|
| Etomidale | 5 | mg |
| Ketamine | 33 | mg |
| Midazolam | 5 | mg |
| Propofol | 50 | mg |

PARALYTIC AGENTS

| | | |
|---|---|---|
| Succinylcholine | 30 | mg |
| Pancuronium | 3.3 | mg |
| Vecuronium | 3.3 | mg |
| Rocuronium | 16 | mg |

MAINTENANCE

| | | |
|---|---|---|
| Pancuronium/Vecuronium | 1.7 | mg |
| Lorazepam | 0.8 | mg |

TABLE 5
Fifth Weight Zone
YELLOW
SEIZURE

| | | |
|---|---|---|
| Lorazepam | 1.3 | mg |
| Diazepam IV | 2.6 | mg |
| Diazepam-RECTAL | 6.5 | mg |
| Phenobarbital Load | 260 | mg |
| Phenytoin Load | 200 | mg |
| Fosphenytoin Load | 200 | mg-PE |

OVERDOSE

| | | |
|---|---|---|
| Dextrose | 6.5 | g |
| Naloxone | 1.3 | mg |
| Flumazenil | 0.13 | mg |
| Glucagon | 0.5 | mg |
| Charcoal | 13 | g |

ICP

| | | |
|---|---|---|
| Mannitol | 13 | g |
| Furosemide | 13 | mg |

FLUIDS

| | | |
|---|---|---|
| Volume Expansion | | |
| Crystalloid (NS or LR) | 260 | mL |
| Colloid, Blood | 130 | mL |
| Maintenance | | |
| DSW + 1/2 NS + 29 | 48 | mL/HR |

Infusion:
Pursuant to JCAHO's
National Patient Safety Goal 3b-
“Rule of 6” for infusions
Should be converted to
Standardized Concentrations.

| Equipment | | |
|---|---|---|
| E.T Tube | 4.5 Uncuffed | |
| E.T Insertion Length | 13.5 | cm |
| Stylet | 6 | French |
| Suction Catheter | 10 | French |
| Laryngoscope | 2 Straight | |
| BVM | Child | |
| Oral Airway | 60 | mm |
| Nasopharyngeal Airway | 20 | French |
| LMA | 2 | |
| O₂ Mask | Pediatric NR8 | |
| ETCO₂ | Pediatric | |
| Urinary Catheter | 10 | French |
| Chest Tube | 20-24 | French |
| NG Tube | 10 | French |
| Vascular Access | 18-22 | Ga |
| Intraosseous | 15 | Ga |

| | |
|---|---|
| BP Cuff | Child |

RESUSCITATION

| Epinephrine (1:10,000) | 0.13 mg | (1.3 mL) |
|---|---|---|
| Epinephrine ET (1:1,000) | 1.3 mg | (1.3 mL) |
| Atropine (0.1 mg/mL) | 0.26 mg | (2.6 mL) |
| Atropine ET (0.4 mg/mL) | 0.4 mg | (1 mL) |
| Sodium Bicarbonate | 13 | mEq |
| Lidocaine | 13 | mg |
| Lidocaine ET | 26-40 | mgs |
| Defibrillation | | |
| 1^{st}/2^{nd} Dose (may repeat) | 26 J/52 J | |
| Cardioversion | | |
| 1^{st}/2^{nd} Dose | 13 J/26 J | |
| Adenosine | | |
| Ist Dose | 1.3 | mg |
| 2^{nd} Dose If Needed | 2.6 | mg |
| Amiodarone | 65 | mg |
| Calcium Chloride | 260 | mg |
| Magnesium Sulfate | 650 | mg |

RAPID SEQUENCE INTUBATION
PREMDICATIONS

| Atropine | 0.25 | mg |
|---|---|---|
| Pan/Vecuronium | N/A | |
| (Defasiculating Agent) | N/A < 20 kg | |
| Lidocaine | 20 | mg |
| Fentanyl | 40 | mcg |

INDUCTION AGENTS

| | | |
|---|---|---|
| Etomidale | 4 | mg |
| Ketamine | 26 | mg |
| Midazolam | 4 | mg |
| Propofol | 40 | mg |

PARALYTIC AGENTS

| | | |
|---|---|---|
| Succinylcholine | 26 | mg |
| Pancuronium | 2.6 | mg |
| Vecuronium | 2.6 | mg |
| Rocuronium | 13 | mg |

MAINTENANCE

| | | |
|---|---|---|
| Pancuronium/Vecuronium | 1.3 | mg |
| Lorazepam | 0.7 | mg |

TABLE 6
Sixth Weight Zone
PURPLE
SEIZURE

| | | |
|---|---|---|
| Lorazepam | 1 | mg |
| Diazepam IV | 2 | mg |
| Diazepam-RECTAL | 5 | mg |
| Phenobarbital Load | 210 | mg |
| Phenytoin Load | 160 | mg |
| Fosphenytoin Load | 160 | mg-PE |

OVERDOSE

| | | |
|---|---|---|
| Dextrose | 5.25 | g |
| Naloxone | 1 | mg |
| Flumazenil | 0.1 | mg |
| Glucagon | 0.5 | mg |
| Charcoal | 10 | g |

ICP

| | | |
|---|---|---|
| Mannitol | 10 | g |
| Furosemide | 10 | mg |

FLUIDS

| | | |
|---|---|---|
| Volume Expansion | | |
| Crystalloid (NS or LR) | 210 | mL |
| Colloid, Blood | 105 | mL |
| Maintenance | | |
| DSW + 1/2 NS + 29 | 43 | mL/HR |

Infusion:
Pursuant to JCAHO's
National Patient Safety Goal 3b-
“Rule of 6” for infusions
Should be converted to
Standardized Concentrations.

| Equipment | | |
|---|---|---|
| E.T Tube | 4.0 Uncuffed | |
| E.T Insertion Length | 11-12 | cm |
| Stylet | 6 | French |
| Suction Catheter | 10 | French |
| Laryngoscope | 1 Straight | |
| BVM | Child | |
| Oral Airway | 60 | mm |
| Nasopharyngeal Airway | 18 | French |
| LMA | 2 | |
| O₂ Mask | Pediatric NR8 | |
| ETCO₂ | Pediatric | |
| Urinary Catheter | 8-10 | French |
| Chest Tube | 16-20 | French |
| NG Tube | 8-10 | French |
| Vascular Access | 20-24 | Ga |
| Intraosseous | 15 | Ga |

| | |
|---|---|
| BP Cuff | Child |

RESUSCITATION

| Epinephrine (1:10,000) | 0.1 mg | (1 mL) |
|---|---|---|
| Epinephrine ET (1:1,000) | 1 mg | (1 mL) |
| Atropine (0.1 mg/mL) | 0.21 mg | (2.1 mL) |
| Atropine ET (0.4 mg/mL) | 0.32 mg | (0.8 mL) |
| Sodium Bicarbonate | 10 | mEq |
| Lidocaine | 10 | mg |
| Lidocaine ET | 20-30 | mgs |
| Defibrillation | | |
| 1^{st}/2^{nd} Dose (may repeat) | 20 J/40 J | |
| Cardioversion | | |
| 1^{st}/2^{nd} Dose | 10 J/20 J | |
| Adenosine | | |
| Ist Dose | 1 | mg |
| 2^{nd} Dose If Needed | 2.1 | mg |
| Amiodarone | 52 | mg |
| Calcium Chloride | 210 | mg |
| Magnesium Sulfate | 525 | mg |

RAPID SEQUENCE INTUBATION
PREMDICATIONS

| Atropine | 0.21 | mg |
|---|---|---|
| Pan/Vecuronium | N/A | |
| (Defasiculating Agent) | N/A < 20 kg | |
| Lidocaine | 15 | mg |
| Fentanyl | 32 | mcg |

INDUCTION AGENTS

| | | |
|---|---|---|
| Etomidale | 3.2 | mg |
| Ketamine | 20 | mg |
| Midazolam | 3.2 | mg |
| Propofol | 32 | mg |

PARALYTIC AGENTS

| | | |
|---|---|---|
| Succinylcholine | 20 | mg |
| Pancuronium | 2.1 | mg |
| Vecuronium | 2.1 | mg |
| Rocuronium | 10 | mg |

MAINTENANCE

| | | |
|---|---|---|
| Pancuronium/Vecuronium | 1 | mg |
| Lorazepam | 0.5 | mg |

One or more of the components and functions illustrated in the FIGS. may be rearranged and/or combined into a single component or embodied in several components without departing from the invention. Additional elements or components may also be added without departing from the invention.

While certain exemplary embodiments have been described and shown in the accompanying drawings, it is to be understood that such embodiments are merely illustrative of and not restrictive on the broad invention, and that this invention is not be limited to the specific constructions and arrangements shown and described, since various other modifications may occur to those ordinarily skilled in the art.

1. A method for determining equipment and medication for resuscitating a pediatric patient, comprising:
determining a mid-point between a shoulder and elbow of an arm of the pediatric patient;
placing a first portion of a measuring tape at the mid-point and wrapping the measuring tape around the arm such that the measuring tape encircles the arm;
identifying a mid-arm circumference of the pediatric patient, the mid-arm circumference identified by a region on the measuring tape that aligns with the first portion when encircling the arm; and
correlating the region with a weight zone, the weight zone identifying medication dosages and equipment sizes for resuscitating the pediatric patient.

2. The method of claim 1, wherein the first portion of the measuring tape has an opening and a second portion of the measuring tape is inserted into and pulled through the opening causing the measuring tape to lay flat on the arm.

3. The method of claim 2, wherein the region is in alignment with the opening.

4. The method of claim 1, wherein the measuring tape includes a plurality of regions identified by a separate color and/or pattern, each region in the plurality of regions corresponding to a different weight zone.

5. The method of claim 1, further comprising:
placing a wrist band around a wrist of the pediatric patient;
identifying the region on the wrist band.

6. The method of claim 1, wherein the measuring tape comprises:
the first portion having a generally rectangular shape;
a second portion having a generally elongated rectangular shape, the second portion including a first end integrally connected to the first portion and a second end; and
an opening, located within the first portion, adapted to receive the second end of the second portion.

7. The method of claim 6, wherein a length of the first portion is approximately one fourth a length of the second portion.

8. The method of claim 6, wherein a height of the first portion is approximately double a height of the second portion.

9. The method of claim 1, wherein the mid-arm circumference is measured while the pediatric patient is standing or sitting.

10. The method of claim 1, wherein the mid-arm circumference is measured while the pediatric patient is lying down.

11. A measuring tape for determining a weight of a pediatric patient, the measuring tape comprising:
a first portion having a generally rectangular shape;
a second portion having a generally elongated rectangular shape, the second portion including a first end integrally connected to the first portion and a second end; and
an opening, located within the first portion, adapted to receive the second end of the second portion;
wherein one side of the second portion includes a plurality of regions identifying different weight zones correlating to weights of pediatric patients; and
wherein each of the different weight zones identifies equipment sizes and medication dosages appropriate for the pediatric patients.

12. The measuring tape of claim 11, wherein the tape determines a mid-arm circumference of the pediatric patient.

13. The measuring tape of claim 12, wherein the mid-arm circumference is measured while the pediatric patient is standing or sitting.

14. The measuring tape of claim 12, wherein the mid-arm circumference is measured while the pediatric patient is lying down.

15. The measuring tape of claim 12, wherein to measure the mid-arm circumference, the first portion is placed at a mid-point between a shoulder and elbow of an arm of the pediatric patient and wrapped around the arm and the second portion of the measuring tape is inserted into and pulled through the opening.

16. The measuring tape of claim 15, wherein each region of the plurality of regions is identified by a separate color and/or pattern

17. The measuring tape of claim 16, wherein the color and/or pattern that are in alignment with the opening identifies a weight zone for the pediatric patient.

18. The measuring tape of claim 11, wherein each region of the plurality of regions is identified by a separate color and/or pattern.

19. The measuring tape of claim 10, wherein a length of the first portion is approximately one fourth a length of the second portion.

20. The measuring tape of claim 10, wherein a height of the first portion is approximately double a height of the second portion.
